(19) United States
(12) Patent Application Publication
(10) Pub. No.: US 2006/0247679 A1
(43) Pub. Date: Nov. 2, 2006

(54) Compression device and method for shape memory alloy implants

(75) Inventor: Marc M. Peterman, Memphis, TN (US)

Correspondence Address: FITZPATRICK CELLA HARPER & SCINTO, 30 ROCKEFELLER PLAZA, NEW YORK, NY, 10112, US

(73) Assignee: SDGI Holdings, Inc., Wilmington, DE (US)

(21) Appl. No.: 11/117,298

(22) Filed: Apr. 29, 2005

(51) Int. Cl. A61B 17/88; A61F 2/46; A61B 17/28; A61F 2/44

(52) U.S. Cl. 606/207; 606/78; 623/17.11; 606/205

(57) ABSTRACT

Apparatus and method for compressing a shape memory material implant to be implanted in a patient. The apparatus in some embodiments includes opposing dies, an actuator, and a uniformity controller. The opposing dies are configured to grasp the shape memory material implant, when placed therebetween. The actuator actuates the opposing dies toward each other to compress the shape memory material implant. The uniformity controller of some embodiments provides uniform compression of a given type of shape memory material implant.

### FIELD OF THE INVENTION

The present invention generally relates to an apparatus for compressing shape memory implants and a method for compressing and implanting such implants. The apparatus and method are generally intended for use in repairing various defects in bone and tissue, such as ruptured or herniated intervertebral discs. More specifically, the present invention may relate to a method and apparatus for compressing shape memory alloy cages in a controllable and repeatable manner.

### BACKGROUND OF THE INVENTION

Implants with shape memory features can be efficacious in repairing bone and tissues defects/deficits. The implants can be used to secure, replace, and/or supplement damaged or missing tissue or bone. The shape memory aspect of the implant typically provides added securement by expanding into the space it is to occupy after an implantation.

A common injury in which such treatment is useful is a ruptured or otherwise damages intervertebral disc. An intervertebral disc is formed of a nucleus of gelatinous collagen fibers and an outer annulus. The gelatinous nucleus provides support between adjacent vertebrae. The nucleus is contained by the annulus, which is formed of layers of collagen fibers which secure the nucleus in place. A common cause of back pain and injury is a rupture or tear in the annulus that allows the nucleus to herniate. A herniated nucleus can put pressure on neural and ligamentary structures associated with the spine, which can leads to pain in the patient's back and legs.

There are numerous conventional treatments for dealing with ruptured or degenerated intervertebral discs. One possible treatment for a ruptured annulus involves repairing the tear in the annulus by inserting an implant into the tear to, essentially, plug the rupture. Thus, the implant may be secured to the annulus at the rupture to help contain the nucleus in its original position. Alternatively, the disc may be completely replaced, or supplemented with a supporting structure. In particular, one treatment for a degenerated disc is to place an implant between adjacent vertebrae to maintain an appropriate space while the vertebrae grow together or fuse.

A favorable option for such procedures is to use a shape memory alloy (SMA) to form the implant. An SMA is an alloy that can be compressed, but will return to an uncompressed form when exposed to certain conditions. For instance, an SMA may be compressed when below a transition temperature, and will expand back to a non-compressed shape when heated above the transition temperature. In other embodiments, the transition may occur through the provision of electrical stimulation, for instance. In addition, similar shape memory materials, such as plastics with shape memory aspects may also be used. For exemplary purposes, however, this application will discuss the present invention with respect to SMAs. One of ordinary skill in the art would understand that alternative materials could be used in the examples discussed below.

Preferably, however, the implant will be formed of an SMA such as nitinol. Nitinol is a mixture of about 50 percent nickel and about 50 percent titanium. By varying the ratio of nickel to titanium, usually only slightly, the transition temperature of the alloy can be adjusted. Also, the SMA will typically be formed in the shape of a cage defined by a lattice structure, an example of which is shown in FIG. 7. With an SMA forming a cage, compression and expansion of the cage is readily achievable. For instance, when below a transition temperature (in situations where the shape memory material is controlled based on temperature), the structure can be compressed to tighten the lattice structure, forming a compressed shape. When heated past the transition temperature, or subjected to such other predetermined condition, the cage will expand back to its original form, or a similar non-compressed form.

An example of such an SMA cage is discussed in U.S. Patent Publication No. US2003/0074075 (Thomas, Jr. et al.). The cage described therein may be used to repair a disc herniation. Another implant is shown in FIG. 7. The implant shown in FIG. 7 may be used as a spacer between adjacent vertebrae to restore intervertebral space lost due to injury or the like. Thus, for example, the implant may support the anterior column load of the spine. Optionally, an osteogenic material may then be packed around the implant.

An SMA cage may be implanted in its compressed form, and then expand to fill the annular defect, intervertebral spaces, or the like. Among other benefits, the implantation of a compressed implant provides for a less invasive procedure, with a smaller incision. Typically, the SMA cage is temperature activated, and the body temperature of the patient heats the SMA cage to above the transition temperature, causing it to expand to the non-compressed form. The transition temperature may be set (for example, by the specific material used to form the cage) at or above 90°, for example, so that the compressed form is stable at room temperature. However, it still can be difficult to keep the cage in the compressed form prior to insertion into a patient. If the product is shipped to the hospital during warmer summer months, the device will likely encounter temperatures during delivery that exceed the transition temperature. This can occur, for instance, in the heat of a delivery truck, exposed to the summer sun. Thus, it is likely that a surgeon, nurse, or technician would have to compress the cage prior to or during the implantation surgery.

Compression of an SMA cage is an important step in a successful implant surgery. If compressed too much, cracking of the material or “cold working” can occur. The lattice is typically arranged in an orderly structure, in both expanded and compressed forms. Cold working occurs when the lattice structure is compressed to a point that crushes or structurally alters the orderly lattice structure. Cold working and cracking of the material can cause the compressed structure to become, at least partially, unrecoverable (“over yielding”), such that the cage does not return to the proper expanded form when heated above the transition temperature.

Typically, about an 8% local material strain is acceptable for an SMA of nitinol. Above that, risks of cracking and cold working heighten. An 8% local material strain could correspond, for example, to a 30% structural deformation of the cage. This, of course, will vary given the specifics of the cage design, material, etc.

Thus, there is a need to provide controllable and repeatable compression of an SMA cage. More specifically, it is beneficial to be able to provide a surgeon with instruments and methods to produce controlled compression of SMA cages, from patient to patient and surgery to surgery and among an array of different types and sizes of SMA cages to be implanted.

### SUMMARY OF THE INVENTION

In one embodiment, the present invention is directed to an apparatus for compressing a shape memory material implant to be implanted in a patient. The apparatus includes opposing dies, an actuator, and a uniformity controller. The opposing dies are configured to grasp the shape memory material implant, when placed therebetween. The actuator actuates the opposing dies toward each other, such that the opposing dies impart forces in substantially direct opposition to each other, against the shape memory material implant, when placed therebetween, so as to compress the shape memory material implant. The uniformity controller provides uniform compression of a given type of shape memory material implant.

In another embodiment, the present invention is directed to a method of inserting a shape memory material implant into a patient. The method includes steps of providing the shape memory material implant, which is compressible and capable of expanding to an expanded form, from a compressed form, when exposed to a predetermined condition, and placing the shape memory material implant in a mechanical actuator with opposing dies, such that the shape memory material implant is grasped between the opposing dies. The mechanical actuator provides controlled, repeatable actuation of the dies. The method also involves actuating the opposing dies of the mechanical actuator toward each other when the shape memory material implant is positioned therebetween, to compress the shape memory material implant into the compressed form, and inserting the compressed shape memory material implant into the patient at a medically efficacious location. After insertion, the compressed shape memory material implant is exposed to the predetermined condition so as to cause the shape memory material implant to expand to the expanded form.

### BRIEF DESCRIPTION OF THE DRAWINGS

FIG. 1 is a side view of a compression device according to an embodiment of the present invention.

FIG. 2 is a side view of an opposite side of the compression device shown in FIG. 1.

FIG. 3 is a front view of the compression device shown in FIG. 1.

FIG. 4 is a side cross section of a portion of the compression device shown in FIG. 3, taken along line 4-4′.

FIG. 5 is a perspective view of a modular die to be used with the compression device shown in FIG. 1.

FIG. 6 is a front view of the modular die shown in FIG. 5.

FIG. 7 is a perspective view of one example of an SMA cage.

FIG. 8 is a perspective view of the SMA cage from FIG. 7 positioned on an insertion device.

FIG. 9 is a side view of a compression device according to another embodiment of the present invention.

### DETAILED DESCRIPTION OF THE INVENTION

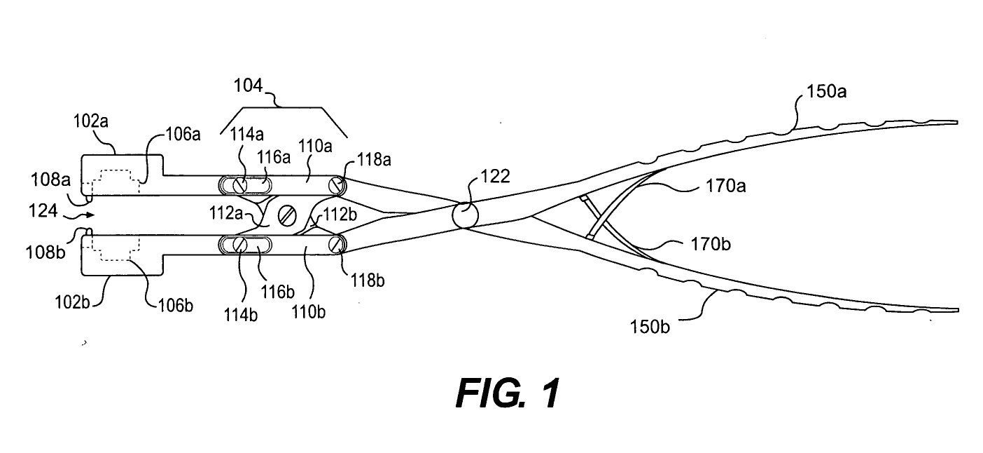

FIGS. 1-3 show one embodiment of the present invention embodied in a hand-held compression device 100. Of course, a number of other arrangements are possible while keeping within the scope of the intended scope of the present invention, as defined by the claims provided below. For instance, the compression device need not be hand held, and can work with alternate mechanics as those described below. For instance, the device may be a table-top device. Also, the mechanics may be automatically controlled through electrical components or pneumatics, such that the device is not actuated manually. Thus, the following description should be taken as exemplary.

As shown in FIG. 1, a compression device 100 is provided. The compression device 100 includes opposing heads 102a and 102b. The opposing heads 102a and 102b include stops 108a and 108b and cavities 106a and 106b. As shown in FIG. 4, in addition to opening towards each other, the cavities 106a and 106b open at distal ends to form window 124. The stops 108a and 108b come into contact when the heads 102a and 102b are actuated toward each other, so as to prevent further actuation after abutment of the stops 108a and 108b.

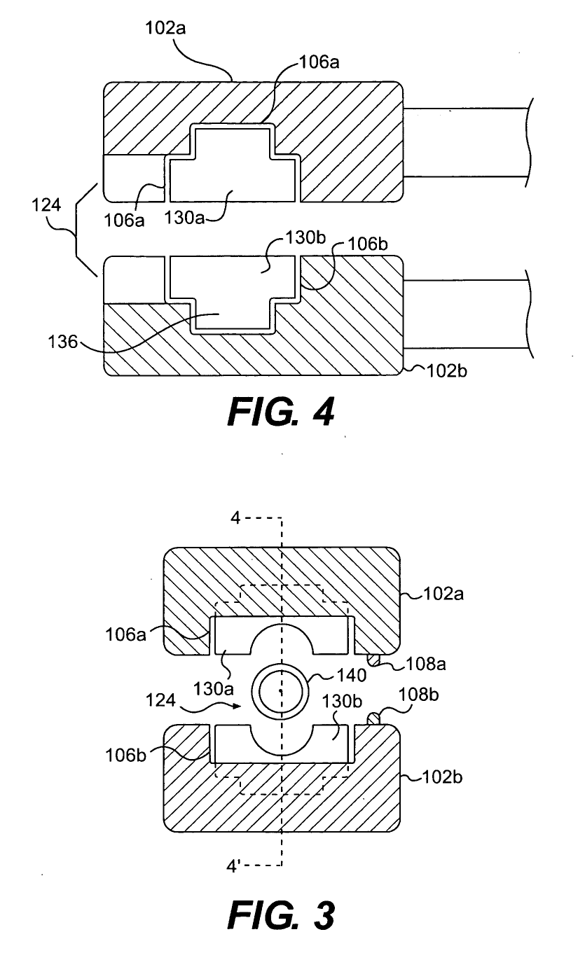

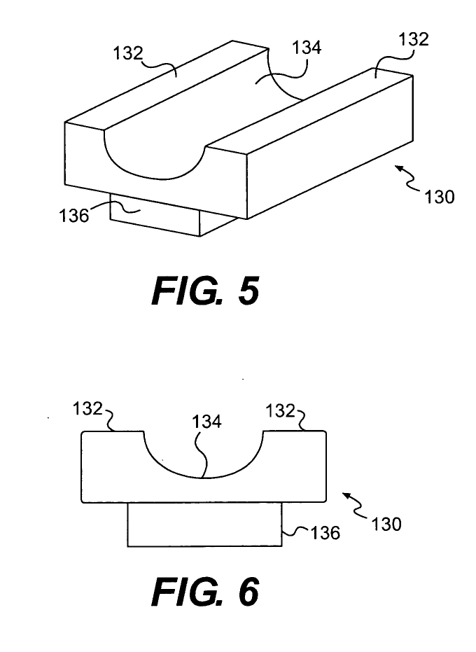

The cavities 106a and 106b each securably receive a die 130 (dies 130a and 130b, are shown in FIGS. 3 and 4). In the depicted embodiment, male and female coupling is provided between the cavities 106a and 106b and the dies 130a and 130b, respectively. In the present embodiment, a male projection 136 of die 130, shown in FIGS. 5 and 6, extends into the depth of the female part of a cavity, such as the cavities 106a and 106b. In this embodiment, a tight fit between the projection 136 and such a cavity secures, for example, the die 130a to the head 102a. However, any one of a number of mechanisms may be used to secure dies 130a and 130b in their respective cavities while keeping within the intended scope of the present invention.

In addition to the projection 136, a die 130 includes die stops 132 and a cradling face 134. In some embodiments of the present invention, the die stops 132 will act to stop further compression of an SMA cage placed between opposing dies 130. In this embodiment, the die stops 132 are flat surfaces of top portions of the die 130 shown in FIGS. 5 and 6. In other embodiments, however, the die 130 may be provided with projections formed integrally with, or secured to, die 130 to act as a die stop. In fact, die stops may be any mechanism that serves to limit the amount of compression applied to a given implant, such as an SMA cage 140 shown in FIG. 3. In the embodiment shown in FIGS. 1-3, die stops 132 will not be used as a stopping mechanism inasmuch as the stops 108a and 108b are provided on the compression device 100, to act as a stopping mechanism to prevent compression of an SMA cage past a given point. It should be appreciated, however, when the stops 108a and 108b are not provided, opposing die stops 132 may project up from the cavities 106a and 106b to abut each other to inhibit further compression.

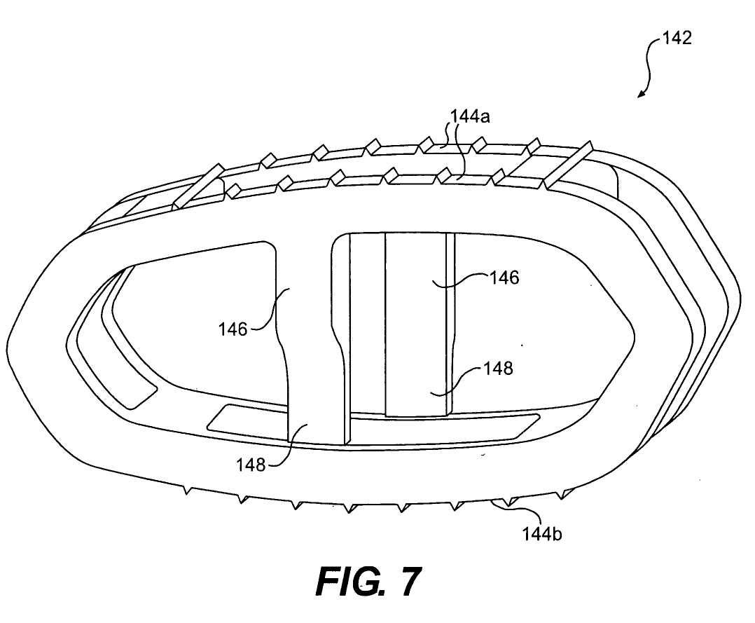

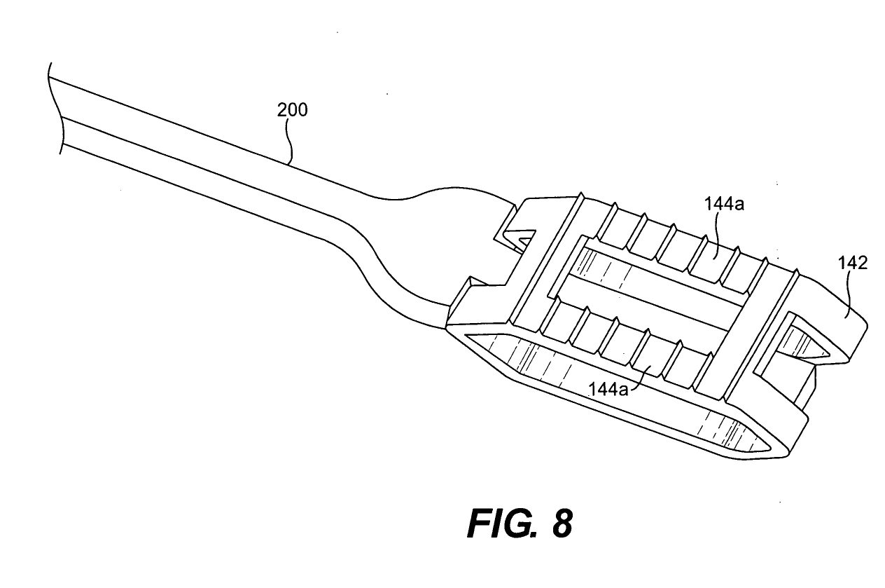

Cradle faces 134 are used to receive and cradle an SMA cage. Specifically, when the dies 130a and 130b are positioned in the heads 102a and 102b, respectively, the cradling faces 134 of the different dies oppose each other so as to receive and cradle an SMA cage therebetween, as shown with respect to the cylindrical SMA cage 140 in FIG. 3. During compression, which occurs by actuating the opposing dies 130a and 130b toward each other in parallel movement to compress an SMA cage positioned therebetween, the cradle faces 134 spread the compression forces across opposite sides of the SMA cage to provide more uniform compression, and to avoid concentrating of a compression force on, for instance, one point of the SMA cage, risking cracking or cold working. In this embodiment, the cradling surfaces 134 are semicylindrical in shape. This shape allows the surfaces of cradling faces 134 to disperse the force of the moving heads 102a and 102b over more of the surface area of the cage. However, the shape of cradling faces 134 may be varied as needed to receive different types of cages. For instance, a less cylindrical SMA cage 142 is shown in FIGS. 7 and 8. Alternative cradling faces may be formed to cradle such a cage. For instance, SMA cage 142 includes support surfaces 144a and 144b, which, when implanted, may contact opposing surfaces of adjacent vertebrae. In the expanded form, shown in FIG. 7, SMA cage 142 acts as a spacer to restore intervertebral space and to bear the anterior column load. Thus, separate cradling faces may be formed to mate with the support surfaces 144a and 144b, so as to compress the SMA cage 142 to move surfaces 144a and 144b closer to each other, prior to implantation. Also, crutches 146 may be manually bent inward before compression of the SMA cage 142, so as not to interfere with or prevent proper compression. Crutches 146 may provide additional strength to the structure when in the expanded form, by moving back into the position shown in FIG. 7. In that position, the crutches 146 may inhibit compression of the cage 142 by acting as a brace between the top and bottom portion of the cage 142, defined by surfaces 144a and 144b, respectively.

However, any one of a number of SMA cages or other compressable devices may be used with the present invention, and SMA cages 140 and 142 are shown only for exemplary purposes.

As discussed earlier, the dies 130a and 130b are removably secured in the heads 102a and 102b. This allows multiple dies 130 to be interchanged in a given compression device 100 so that the compression device 100 can be used with a variety of different types and sizes of SMA cages. In some embodiments, compression device 100 will be provided with a set of interchangeable dies 130, which correspond with SMA cages of different sizes and styles. In other embodiments, a die 130 may be provided with a particular SMA cage to be implanted, so as to account for design changes over time. Thus, the dies 130 may be made specific to the SMA cages with which they are to be provided.

The dies 130 can be made of any one of a number of different types of materials. For example, plastics such as acetyl copolymer or polyethylene may be used. Plastics are beneficial because they are not as hard as metal, and thus are less likely to damage the implants. Of course any one of a number of types of materials may be used, including metals.

Actuation Control

When moving the heads 102a and 102b together to provide compression, as discussed above, dies 130a and 130b may move in parallel such that the dies 130a and 130b provide substantially opposing forces against an SMA cage positioned therebetween. For instance, if a cage is shaped as a cylinder, opposing dies 130a and 130b may provide forces in substantially opposing radial directions. By providing substantially opposing forces, it is possible to help prevent shearing forces that could crack or otherwise damage the SMA cage. In addition, this helps prevent uneven compression of the lattice structure of a given SMA cage, particularly in connection with the shape of the cradling surface. Thus, the substantially opposing forces may be applied in opposition to each other substantially along (i.e., with respect to) a single axis of the implant to be compressed, or a common axis of the opposing dies. The axis can be any straight line (i) passing through the implant positioned in the compression device 100, or (ii) passing through both opposing dies. When the implant is substantially rectangular in shape, the forces may be described as being applied to opposite transverse or opposite lateral surfaces of the implant.

Any mechanism may be used to provide such even compression to avoid shearing forces and the like. Such mechanisms may include gears or levers that operate to move the dies 130 to provide such opposing forces. In addition, it is possible that only one die 130 moves, while an opposing die is kept stationary (either completely, or partially to incorporate a rotational aspect such as with a gimble support or the like) such that the opposing die provides a resistance force during compression. For example, the mechanics of a conventional die press machine may be incorporated into the present invention to provide the actuation.

For exemplary purposes, we show a hand-held compression device 100. One of ordinary skill in the art would recognize that the design thereof can be varied as discussed above or in other manners to provide the forces necessary for compression. In the present compression device 100, handles 150a and 150b are operated by a user to provide force to actuate the heads 102a and 102b. As shown in FIG. 1, the compression device 100 may also be provided with springs 170a and 170b which provide a biasing force to keep the handles 150a and 150b in an open position when not in use. While these springs are shown, other biasing mechanisms may be used while keeping within the scope of the present invention. In addition, the springs 170a and 170b are provided only for ease of use and are not necessary for operation.

Also, with respect to the compression device 100, to keep the heads 102a and 102b moving in parallel, a four-bar linkage 104 is used in this embodiment. The four-bar linkage 104 includes parallel bars 110a and 110b and crossing bars 112a and 112b. The heads 102a and 102b are secured to the parallel bars 110a and 110b, respectively. The crossing bars 112a and 112b have a common pivot point defined by a post 120. The crossing bar 112a is pivotably connected to the parallel bar 110a at a common pivot point defined by a post 118a. The crossing bar 112a is also pivotably connected to the parallel bar 110b by a post 114b. The crossing bar 112b is pivotably connected to the parallel bar 110a by a post 114a. The crossing bar 112b is also pivotably connected to the parallel bar 110b by a post 118b.

In addition, the posts 114a and 114b, secured to the crossing bars 112a and 112b, respectively, slide relative to the parallel bars 110a and 110b, respectively, within slots 116a and 116b, which are formed in the parallel bars 110a and 110b.

The handle 150b crosses, and is pivotably secured to, the handle 150a by a post 122. The handle 150b is pivotably connected to the parallel bar 110a at post 118a. Handle 150a is similarly connected to the parallel bar 110b at post 118b.

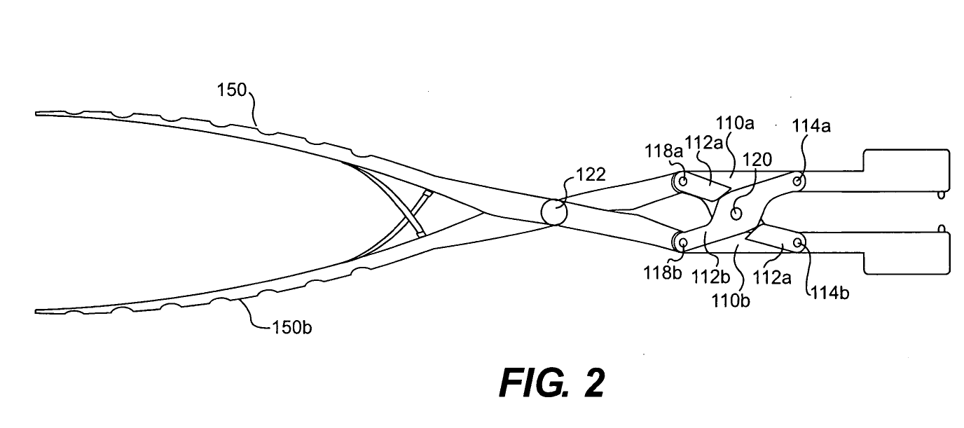

Thus, as the handles 150a and 150b are moved from an open position, at which they are spaced apart from each other, to a closed position (i.e., toward each other), parallel bars 110a and 110b are also biased toward each other. As the parallel bars 110a and 110b are biased toward each other, they pivot with respect to handles 150b and 150a, respectively, about posts 118a and 118b, respectively. In addition, as the parallel bars 110a and 110b are biased toward each other, the crossing bars 112a and 112b pivot about posts 118a, 120, and 114b, and 114a, 120 and 118b, respectively, so as to move from a position defined by a substantial “X” shape (shown in FIG. 2) made by those two bars, to a position in which the “X” flattens as the crossing bars 112a and 112b rotate toward more parallel positions.

As the crossing bars 112a and 112b pivot with respect to each other and the parallel bars 110a and 110b, posts 114b and 114a, secured thereto, respectively, slide within slots 116a and 116b. In this regard, we note that the length of the slots 116a and 116b can be varied in accordance with design choices. When made shorter, slots 116a and 116b can form stops that inhibit further actuation of the compression device 100, and specifically, actuation of opposing dies 130a and 130b toward each other.

With such action, as the handles 150a and 150b actuate the parallel bars 110b and 110a together, parallel bars 110b and 110a remain substantially parallel with each other. Because the heads 102a and 102b are secured to the parallel bars 110a and 110b, heads 102a and 102b actuate in parallel while moving towards to each other, so as to compress an SMA cage positioned between the dies 130a and 130b. This parallel movement helps prevent shearing forces that could damage a cage during compression. Thus, the opposing dies 130a and 130b each have substantially opposing movement along an axis common to dies 130a and 130b, with the directions of movement of each being in substantially opposing radial directions of SMA cage 140 shown in FIG. 3, for example.

Again, however, this is only one mechanism for providing actuation of dies 130a and 130b. Numerous other arrangements may be used to provide adequate opposing forces to a given SMA cage during compression.

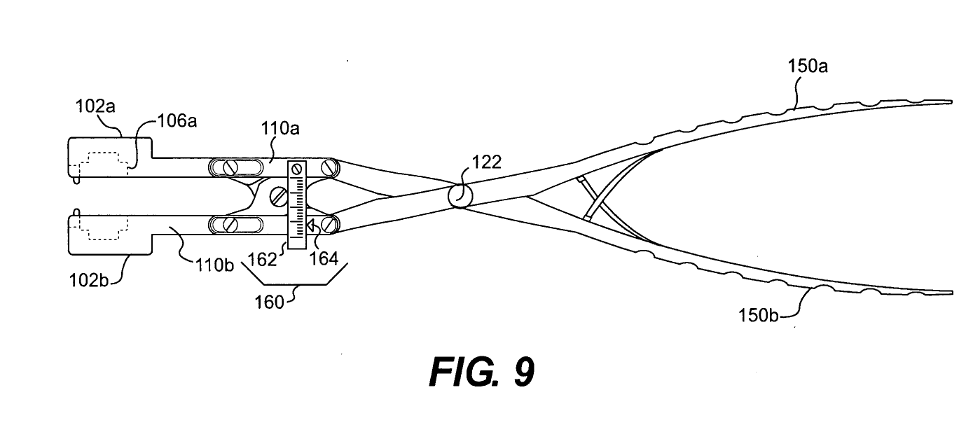

In the embodiments shown in FIG. 9, compression device 100 is provided with a graduated scale 160. The graduated scale 160 includes a graduation plate 162 and a pointer 164. The gradation plate 162 is secured to parallel bar 110a, and moves freely with respect to parallel bar 110b. The parallel bar 110b has provided thereon the pointer 164, which points to the graduation plate 162, to indicate a graduation mark thereon. As the parallel bars 110a and 110b are actuated, the pointer 164 and the graduation plate 162 move relative to each other. Thus, the pointer 164 can indicate a mark corresponding to a beginning point of compression and a mark corresponding to an ending point of compression, so as to aid in controlled and repeatable compression amounts.

The manufacturer of a given type of SMA cage can indicate a preferred compression amount or compression range for a specific SMA cage, which a user of the compression device 100 can measure using the graduated scale 160. In that manner, like mechanical stops, the graduated scale 160 acts as a mechanism for inhibiting compression past a given compression amount (in association with presumed user vigilance) to help prevent over compression of an SMA cage. Unlike a stop, inhibition of over-compression is provided by a user operating the device so as to provide a given compression amount as indicated by the graduated scale 160. Of course, the compression level does not have to be specifically indicated by the manufacturer, and the graduated scale 160 can be used to keep track of a compression amount of an SMA cage dictated by a user of the compression device 100.

Example Implantation Method

With a compression device according to the present invention, the implantation of an SMA cage, such as the SMA cage 140 or SMA cage 142, can be controlled and repeatable, leading to improved implantation techniques. In connection with such a compression device or other compression devices, another embodiment of the present invention is a preferred method of implanting SMA cages, or similar shape memory implants.

With respect temperature sensitive SMA cages, compression is more easily achieved at lower temperatures, i.e., temperatures further from the transition temperature of the SMA. Thus, one embodiment, an implant surgery for inserting or securing the SMA cage 140 (for example) in a patient will involve reducing the temperature of the SMA cage 140 prior to implantation. The method of doing this may involve submerging, completely or partially, the SMA cage 140 in an ice bath. The SMA cage 140 may be submerged by plunging it directly into the ice bath (if sterile), or plunging in a sterile packet containing the SMA cage 140, to maintain a sterile field during surgery.

Once the SMA cage 140 is sufficiently reduced in temperature, it can be removed from the ice bath. The amount of temperature reduction can be varied as needed, depending on the transition temperature of the SMA, compression amount necessary, etc., as would be understood by one of ordinary skill in the art.

If the compression device being used includes modular dies, proper modular dies would be selected in view of manufacturer suggestions, dies provided with SMA cage 140, or in accordance with the surgeon's own judgment. To insert a selected die, for instance, the compression device 100 could be opened to allow room for insertion of the dies 130a and 130b. The dies 130a and 103b should be inserted and secured. Once secured, SMA cage 140 may be inserted into the compression device 100 through the window 124, so as to be positioned between opposing cradling surfaces 134. (When an implant such as the SMA cage 142 is used, the implantation method may include a step of manually biasing free ends 148 of crutches 146 inward (or outward), to allow for proper compression. In re-expansion, crutches 146 will reposition automatically to add strength to the SMA cage 142.) In this embodiment, the handles 150a and 150b are squeezed to actuate the dies 130a and 130b until the opposing surfaces 134 just contact SMA cage 140, simultaneously. In other words, the handles may be moved to the closed position until the opposing dies 130a and 130b just grip SMA cage 140 therebetween so as to cradle the SMA cage 140 simultaneously with opposing cradling surfaces 134.

At this point, if the compression device 100 includes a graduated scale 160, an initial reading of the graduated scale 160 may be taken to determine the starting point of compression. When compression is based on the manufacturer's provided compression amount, to be measured by a scale such as graduated scale 160, a user squeezes the handles 150a and 150b to actuate the heads 102a and 102b to compress the SMA cage 140 until the indicated compression is achieved, as measured by the movement of pointer 164 with respect to the graduation plate 162.

In other embodiments, the handles 150a and 150b may be squeezed until further compression is inhibited by a stopping mechanism. For instance, further compression may be inhibited by abutment of the stops 108a and 108b, or the die stops 132. Of course, other stopping mechanisms may be provided, as would be understood by one of ordinary skill in the art. Also, the compression may be stopped based on the user's judgement.

Before compression is complete, an insertion device may be positioned so as to be secured to the SMA cage to be used. For instance, as shown in FIG. 8, when a tip of the insertion device 200 is placed inside the SMA cage 142, as compression continues, the SMA cage 142 will be clamped onto the insertion device 200. This allows for ease of (i) removal of the SMA cage 142 from compression device 100 and (ii) insertion into the patient.

Once the desired compression of the SMA cage 140 is achieved, the handles 150a and 150b may be released. When the springs 170a and 170b, or other such springs, are provided with respect to handles 150a and 150b, release of the handles 150a and 150b will be followed by automatic biasing of the handles 150a and 150b to the open position. In the open position, the compressed SMA cage 140 can be removed through the window 124.

When the surgeon, nurse, or technician moves the SMA cage 140 from the compression device 100, it can be achieved by using a user's hand(s) or by using a sterile insertion device, such as insertion device 200.

The compressed SMA cage 140 can be implanted directly into an area to be treated. This can be achieved by the surgeon directly implanting compressed SMA cage 140, or by inserting the compressed SMA cage 140 into position using insertion device, such as the insertion device 200.

The body heat of the patient will heat the compressed SMA cage 140 above the transition temperature (in instances in which temperature activated SMAs are used), causing compressed SMA cage 140 to expand to its expanded form. At this point, the cage will release its grip on an insertion device being used (such as insertion device 200) and the insertion device can be removed. This expansion further secures the SMA cage 140 in the implantation area. Of course, the application of some other stimulus may be provided to the compressed SMA cage 140 or other shape memory material, when the material is not temperature activated. Once the compressed SMA cage 140 is fully or partially secured in position, the surgeon may close the patient.

Thus, with the compression device 100, a surgeon can achieve controlled and repeatable compression, providing uniformity from surgery to surgery and preventing the likelihood of cracking of the SMA cage or cold working, which could lead to a defective implant. Thus, implantation of SMA cages can be improved so as to be more reliable, and thus more effective over a wide array of instances.

While the present invention has been described with respect to what is presently considered to be example embodiments, the present invention is not limited to the disclosed embodiments. Rather, the present invention covers various modifications and equivalent arrangements included within the spirit and scope of the appended claims. The scope of the appended claims is to be accorded the broadest interpretation so as to encompass all such modifications and equivalent structures and functions.

I claim:

1. An apparatus for compressing a shape memory material implant to be implanted in a patient, comprising:
opposing dies configured to grasp the shape memory material implant, when placed therebetween;
an actuator for actuating said opposing dies toward each other such that said opposing dies impart forces being in substantially direct opposition to each other, against the shape memory material implant, when placed therebetween, so as to compress the shape memory material implant; and
a uniformity controller for providing uniform compression of a given type of shape memory material implant.

2. An apparatus according to claim 1, wherein said uniformity controller comprises a mechanical stop for inhibiting further movement of said opposing dies towards each other past a set distance therebetween.

3. An apparatus according to claim 1, wherein said actuator comprises a lever mechanism for imparting the opposing forces against the shape memory material implant.

4. An apparatus according to claim 1, wherein each of said opposing dies comprises a concave surface, with said concave surfaces configured to cradle the shape memory material implant.

5. An apparatus according to claim 3, wherein said apparatus comprises two handles pivotably intersecured to rotate about a common axis, the pivotably intersecured handles forming said lever mechanism, and said two handles are integrated with said opposing dies, respectively, such that movement of said handles actuates said opposing dies to impart the opposing forces against the shape memory material implant, when moved from an open position to a closed position.

6. An apparatus according to claim 5, wherein said opposing dies are positioned opposite the common axis from said two handles.

7. An apparatus according to claim 6, wherein said concave surfaces of said opposing dies are each substantially semicylindrical in shape, with longitudinal axes of said semicylindrical concave surfaces being arranged substantially perpendicular to the common axis of said handles.

8. An apparatus according to claim 7, wherein longitudinal ends of said semicylindrical concave surfaces distal to the common axis of said handles define a window in which the shape memory material implant is inserted into said apparatus when said handles are in the open position.

9. An apparatus according to claim 2, wherein said opposing dies are modular opposing dies removably secured to said apparatus.

10. An apparatus according to claim 9, wherein said mechanical stop is integrated with at least one of said modular opposing dies.

11. An apparatus according to claim 2, wherein said actuator comprises a four-bar linkage interconnected with said opposing dies so as to operate said opposing dies in parallel to impart the substantially opposing forces.

12. An apparatus according to claim 1, wherein said uniformity controller comprises a gauge for indicating to a user of said apparatus the amount of relative movement between said opposing dies.

13. An apparatus according to claim 12, wherein each of said opposing dies comprises a concave surface, with said concave surfaces configured to cradle the shape memory material implant.

14. An apparatus according to claim 12, wherein said gauge comprises a graduated scale for measuring an actuation amount of said opposing dies during compression of a shape memory material implant.

15. An apparatus according to claim 12, wherein said opposing dies are modular opposing dies removably secured to said apparatus.

16. An apparatus according to claim 12, wherein said actuator comprises a four-bar linkage interconnected with said opposing dies so as to operate said opposing dies in parallel to impart the substantially opposing forces.

17. A method of inserting a shape memory material implant into a patient, comprising the steps of:
providing the shape memory material implant which is compressible and capable of expanding to an expanded form, from a compressed form, when exposed to a predetermined condition;
placing the shape memory material implant in a mechanical actuator with opposing dies, such that the shape memory material implant is grasped between the opposing dies, wherein the mechanical actuator provides controlled, repeatable actuation of the dies;
actuating the opposing dies of the mechanical actuator toward each other when the shape memory material implant is positioned therebetween, to compress the shape memory material implant into the compressed form;
inserting the compressed shape memory material implant into the patient at a medically efficacious location; and
exposing the compressed shape memory material implant to the predetermined condition so as to cause the shape memory material implant to expand to the expanded form.

18. The method according to claim 17, wherein the mechanical actuator is hand operated such that said actuating step involves hand actuating the opposing dies.

19. The method according to claim 17, wherein the mechanical actuator comprises a graduated scale for measuring a compression level, and said actuating step involves actuating the opposing dies to compress a given shape memory material implant positioned therebetween to a specified level, using the graduated scale to measure the relative movement of the opposing dies.

20. The method according to claim 17, wherein the predetermined condition is a temperature above a transition temperature, and said method further comprises the step of reducing the temperature of the shape memory material implant to ensure that the shape memory material implant is below the transition temperature, prior to said actuating step, wherein the body temperature of the patient heats the shape memory material implant above the transition temperature so as to cause the shape memory material implant to expand to the expanded form, after said inserting step.

21. The method according to claim 19, wherein the opposing dies are modular, and said method further comprises steps of selecting a pair of modular dies to be used with a given shape memory material implant, and securing the selected pair of modular dies in the mechanical actuator prior to said step of placing the reduced temperature shape memory material implant in the mechanical actuator.

22. The method according to claim 19, wherein said actuating step comprises the sub-steps of:
i) actuating the opposing dies to compress the shape memory material implant along a first axis of the shape memory material implant;
ii) rotating the compressed shape memory material implant; and
iii) actuating the opposing dies again to compress the shape memory material implant along a second axis of the shape memory material implant.

23. The method according to claim 21, wherein the opposing dies are modular, and different sets of dies are used for compression along the first and second axes, respectively.

24. An apparatus for compressing a shape memory material implant to be implanted in a patient, comprising:
compression means for compressing the shape memory material implant when placed in said compression means, from an expanded form to a compressed form;
actuation means for actuating said compression means from an open position, in which said shape memory material implant can be inserted into said compression means, to a closed position in which said compression means compresses said shape memory material implant; and
inhibition means for inhibiting said compression means to compress said shape memory material implant past a specified compression level.

25. The apparatus according to claim 24, wherein said inhibition means comprises a mechanical stop positioned to prevent further actuation of said compression means past the specified compression level.

26. The apparatus according to claim 24, wherein said inhibition means comprises a gauge for indicating to a user a level of compression applied to a shape memory material implant compressed by said compression means, so that the user may inhibit compression past the specified compression level.
